(19) United States
(12) Patent Application Publication
(10) Pub. No.: US 2008/0234323 A1
(43) Pub. Date: Sep. 25, 2008

(54) Amorphous and Three Crystalline Forms of Rimonabant Hydrochloride

(76) Inventors: Braj Bhushan Lohray, Gujarat (IN); Vidya Bhushan Lohray, Gujarat (IN); Bipin Pandey, Gujarat (IN); Mayank Ghanshyambhai Dave, Gujarat (IN); Parind Narendra Dholakia, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 11/794,264

(22) PCT Filed: Jan. 6, 2006
(86) PCT No.: PCT/IN06/00006

§ 371 Date: Apr. 28, 2008

(30) Foreign Application Priority Data
Jan. 6, 2005 (IN) 15/MUM/2005

(51) Int. Cl. A61K 31/454; C07D 401/12

(52) U.S. Cl. 514/326; 546/211

(57) ABSTRACT

The present invention describes novel forms of Rimonabant hydrochloride, processes for their preparation and pharmaceutical compositions containing them. Thus, the present invention discloses three new crystalline forms designated as Form II, Form III and Form IV of Rimonabant hydrochloride and novel amorphous forms of the salt.

### FIELD OF INVENTION

The present invention describes novel forms of Rimonabant hydrochloride, processes for their preparation and pharmaceutical compositions containing them. The present invention also describes method of treatment of obesity, smoking cessation, overweight and related diseases comprising administration to a patient in need of such treatment of a non-toxic therapeutically effective amount of the said novel polymorphs and pharmaceutical composition containing them. The present invention relates to the use of novel polymorphs of Rimonabant hydrochloride disclosed herein and pharmaceutical compositions containing them for the treatment of obesity, smoking cessation, overweight and related diseases.

### BACKGROUND OF THE INVENTION

Rimonabant is one of the potentially newer therapies discovered for the treatment of obesity, smoking cessation, overweight and related diseases, currently undergoing Phase-III clinical trials.

Rimonabant is 5-(4-chlorophenyl)-1-(2,4-dichlorophenyl)-4-methyl-N-(piperidin-1-yl)pyrazole-3-carboxamide, having structural formula I.

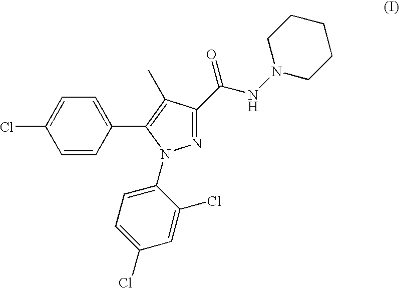

It is presently being developed by Sanofi as its hydrochloride salt, as a CB₁ antagonist, as a potential treatment for obesity, smoking cessation, Alzheimer's disease, Parkinson's disease etc. This compound was first disclosed in EP 656354 and also in U.S. Pat. No. 5,624,941 which is hereby incorporated by reference in its entirety.

The therapeutic applications of Rimonabant has been described in U.S. Pat. No. 6,344,474, U.S. Pat. No. 6,642,258, WO 0158450, WO 0185092, WO 0318060, WO 0382256 etc. which are also incorporated in their entirety as reference.

WO 03040105 (Sanofi) and US 20050043356 discloses one new crystalline form of Rimonabant base designating it as Form II of the compound, which is also hereby incorporated as reference in its entirety.

U.S. Pat. No. 5,624,941 (Sanofi) discloses the HCl salt of Rimonabant having a melting point of 224° C. The method claimed in this patent allows the preparation of Rimonabant hydrochloride in crystalline form which will be called as Form 1.

However, the present inventors have found that the crystalline form of Rimonabant hydrochloride disclosed in the above mentioned application is difficult to purify, difficult to reproduce by the process described in U.S. Pat. No. 5,624,941. Hence, the present inventors felt the need to develop such forms, which are very stable, reproducible and easy to formulate. It has now been surprisingly found out that Rimonabant hydrochloride can exist in different polymorphic crystalline forms which differ from each other in their stability, in their physical properties, in their spectral characteristics and in their methods of preparation. Surprisingly, these new forms were found to be easy to purify, are easily reproducible and can be formulated easily. Moreover, it was found that the crystalline forms of the present invention were stable making them suitable for use as pharmaceutically acceptable products.

It is normally accepted that the amorphous form of any compound are less desirable due to obvious problems in formulating an amorphous solid. Moreover, such forms have the problem of stickiness due to moisture absorption, very high solubility and other associated problems. However, it was surprisingly found that one of the amorphous form of Rimonabant hydrochloride of the present invention was very stable over long term. The enhanced stability of the amorphous form of the present invention may be probably due to the presence of water molecule associated with the salt. Preliminary studies indicate that such amorphous form may be suitable for preparation of intravenous and/or injectable formulation of the drug, which will have significant therapeutic applications. Such novel formulations may be tried out for this drug looking at the type of diseases for which the drug is indicated. It has also been found surprisingly that one of the amorphous form of the present application can be used to obtain the different forms of Rimonabant hydrochloride in pure form.

The present invention thus discloses amorphous forms and three new crystalline forms of Rimonabant hydrochloride designated as Form II, Form III & Form IV respectively.

### SUMMARY OF THE INVENTION

Accordingly, the present invention provides new crystalline forms of Rimonabant hydrochloride or mixture thereof.

In another embodiment of the present invention is provided novel amorphous forms of Rimonabant hydrochloride.

In a further embodiment is provided processes for the preparation of the novel forms of Rimonabant hydrochloride or mixture thereof.

Yet in another embodiment is provided pharmaceutical compositions comprising the said novel forms of Rimonabant hydrochloride.

In a still further embodiment is provided uses of the novel forms of Rimonabant hydrochloride or the treatment of obesity, Parkinson's disease, Alzheimer's disease, smoking cessation and other related diseases.

### BRIEF DESCRIPTION OF THE ACCOMPANYING DRAWINGS

FIG. 1: X-Ray powder diffraction (XRD) pattern of amorphous form of Rimonabant hydrochloride.

FIG. 2: X-Ray powder diffraction (XRD) pattern of novel crystalline form II of Rimonabant hydrochloride.

FIG. 3: X-Ray powder diffraction (XRD) pattern of novel crystalline form III of Rimonabant hydrochloride.

FIGS. 4 & 5: X-Ray powder diffraction (XRD) pattern of novel crystalline form IV of Rimonabant hydrochloride.

### DESCRIPTION OF INVENTION

Rimonabant is (I) 5-(4-chlorophenyl)-1-(2,4-dichlorophenyl)-4-methyl-N-(piperidin-1-yl)pyrazole-3-carboxamide, having structural formula (I).

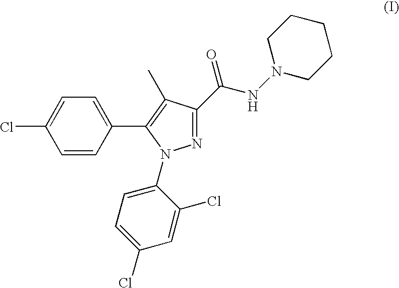

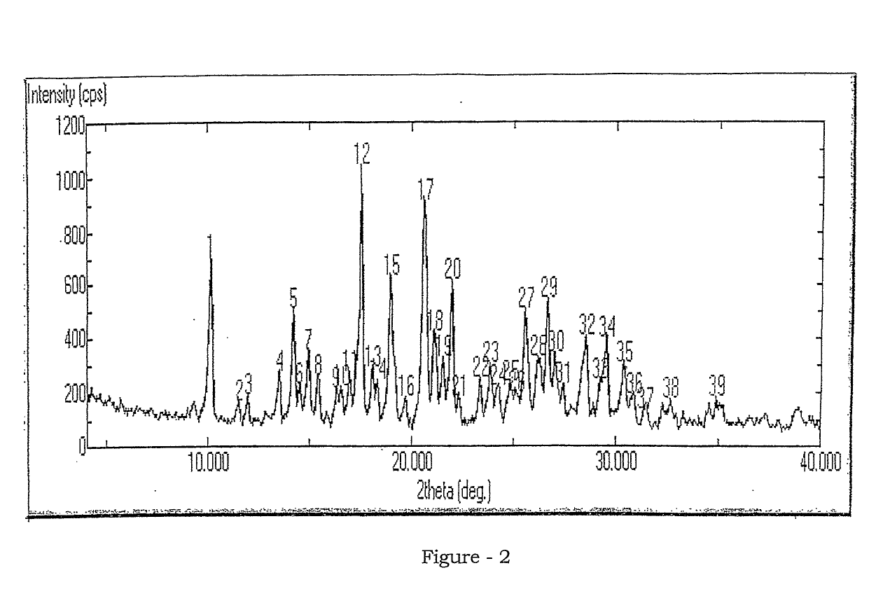

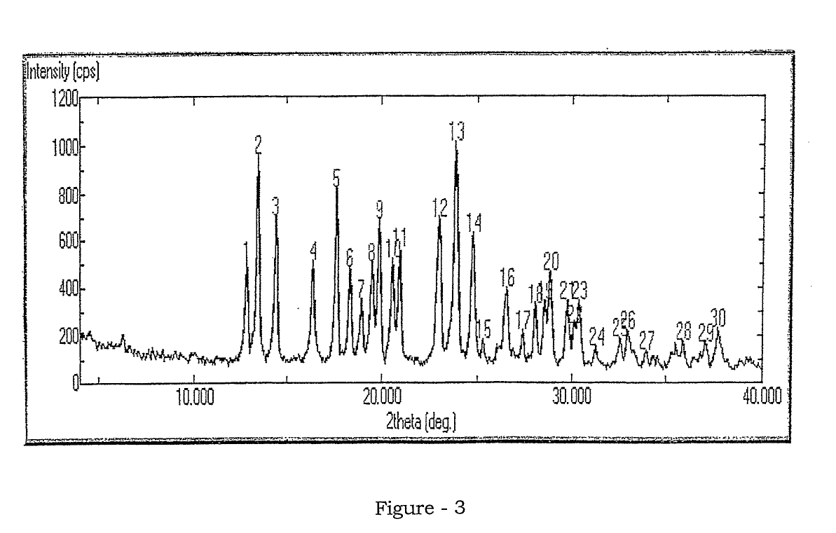

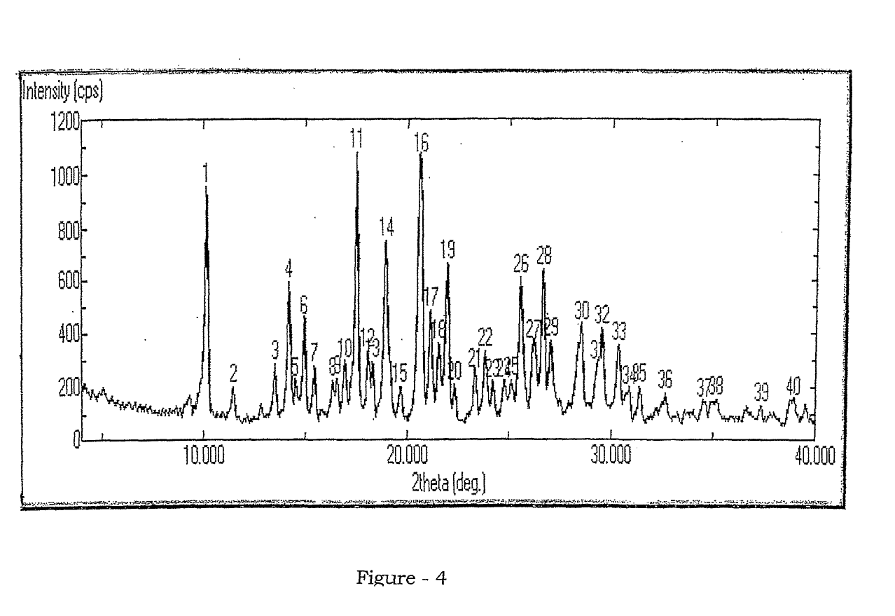

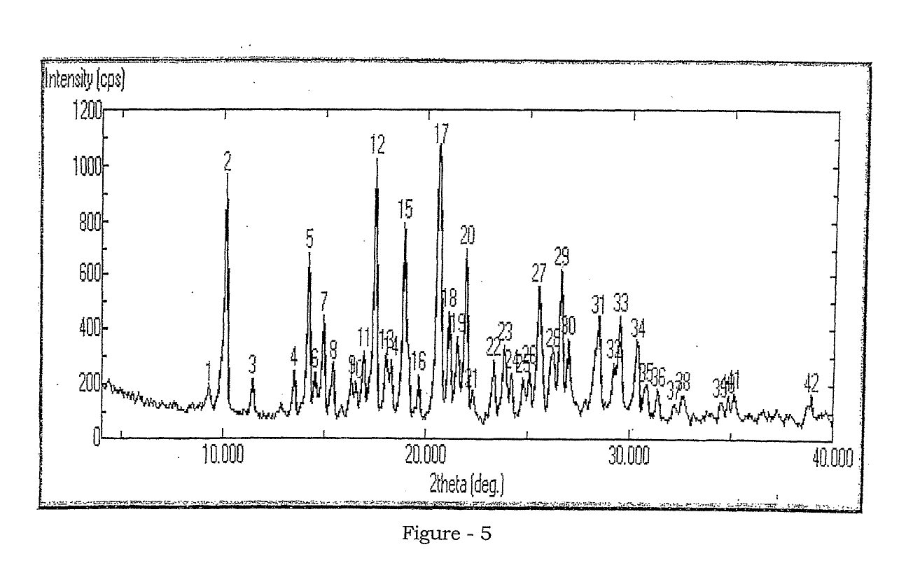

The present invention provides novel forms of Rimonabant hydrochloride as given below:
i) Amorphous forms;
ii) Crystalline Form II of Rimonabant hydrochloride having melting point in the range of 237-244° C., having characteristic XRD pattern as provided in FIG. 2.
iii) Crystalline Form III of Rimonabant hydrochloride having melting point in the range of 240-245° C., having characteristic XRD pattern as provided in FIG. 3.
iv) Crystalline Form IV of Rimonabant hydrochloride having melting point in the range of 242-247° C., having characteristic XRD pattern as provided in FIG. 4.

The novel forms of Rimonabant hydrochloride are characterized by unique XRD patterns which are different from the various forms reported in the above mentioned applications.

The present invention also discloses processes for the preparation of the said novel forms of Rimonabant hydrochloride and pharmaceutical compositions containing them and their use in medicine. The general processes for preparing the various novel forms of the present invention are provided below. It will be appreciated that a skilled person may modify/alter these processes suitably in an obvious manner and such obvious alternations/modifications are considered included within the scope of the present application.

### I) Preparation of Amorphous Form of Rimonabant Hydrochloride

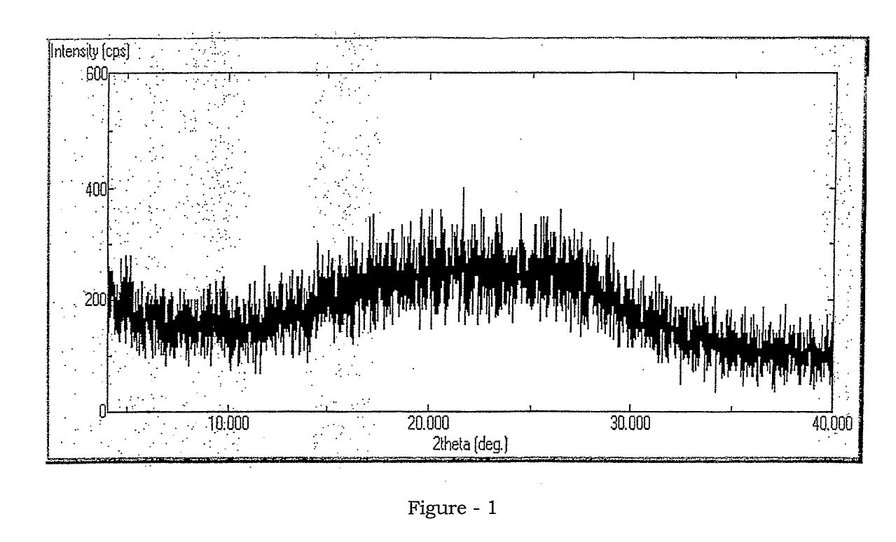

The novel amorphous form of Rimonabant hydrochloride may be prepared by dissolving/contacting Rimonabant base in suitable solvents selected from (C₁-C₆) alcohols such as methanol, ethanol, propanol, n-butanol and the like, benzene, dichloromethane, dichloroethane, acetone, cyclohexane, dimethyl formamide, dimethyl acetamide, 1,4-dioxane, tetrahydrofuran or mixtures thereof and treating with dilute HCl, stirring the solution, filtering and controlled drying the residue to obtain amorphous form of Rimonabant hydrochloride, having XRD pattern as provided in FIG. 1.

### II) Preparation of Crystalline Form II of Rimonabant Hydrochloride

The amorphous form of Rimonabant hydrochloride was stirred in ethyl acetate, suitable ethers such as diethyl ether, methyl t-butyl ether (MTBE), diisopropyl ether and the like or mixtures thereof, filtered and subsequently removing the solvent from the solid obtained to get the novel crystalline form II of Rimonabant hydrochloride, m. p. −237-244° C. and having characteristic XRD pattern, as provided in FIG. 2.

### III) Preparation of Crystalline Form III of Rimonabant Hydrochloride

The amorphous form of Rimonabant hydrochloride was stirred in isopropanol, filtered and subsequently removing the solvent from the solid obtained to get the novel crystalline form III of Rimonabant hydrochloride, m. p. −240-245° C. and having characteristic XRD pattern as provided in FIG. 3.

### IV) Preparation of Crystalline Form IV of Rimonabant Hydrochloride

The amorphous form of Rimonabant hydrochloride was stirred in (C₅-C₁₂) alcohols such as pentanol, isopentanol, hexanol, heptanol, decanol, undecanol and the like, acetone or mixtures thereof, filtered and subsequently removing the solvent from the solid obtained to get the novel crystalline form IV of Rimonabant hydrochloride, m.p. 242-244° C. and having characteristic XRD pattern as provided in FIGS. 4 and 5.

The various pharmaceutical compositions and formulations of the novel forms of Rimonabant hydrochloride of the present invention can be prepared by processes well known. The dosage of the novel forms of Rimonabant hydrochloride of the present invention is selected according to the usage and may vary as per the requirement of the patient.

Pharmaceutical compositions of the present invention contains amorphous Rimonabant hydrochloride and new crystalline forms II, III and IV of Rimonabant hydrochloride, optionally in mixture with other form(s) or amorphous Rimonabant hydrochloride as active ingredients. Rimonabant hydrochloride forms II, III, IV and amorphous form obtained by the processes of the present invention are ideal for pharmaceutical composition in that they have the purity of at least about 90%, more preferably at least about 95% and most preferably at least about 99%. In addition to the active ingredient(s), the pharmaceutical composition of the present invention may contain one or more excipients. Excipients are added to the composition for preparing various dosage forms using the techniques and processes known.

The novel forms of Rimonabant hydrochloride of the present invention may be used for the treatment of obesity, Parkinson's disease, Alzheimer's disease, smoking cessation and other related diseases in a mammal including human.

The process described in the present invention is illustrated in the following examples which are provided for illustration only and should not be construed to limit the scope of the invention in any way.

### EXAMPLE 1

### Preparation of Amorphous Form of Rimonabant Hydrochloride

Rimonabant base 5 g was dissolved in methanolic HCl solution till acidic pH, followed by addition of water and the reaction mixture was stirred at room temperature. The solvent was distilled off under reduced pressure to get a sticky solid mass. Methanol was added and again solvent was distilled off to get the desired solid. XRD pattern showed the amorphous form of the compound.

% water: 3-5% (different batches)

### EXAMPLE 2

### Preparation of Amorphous Form of Rimonabant Hydrochloride

Rimonabant hydrochloride 5 g was dissolved in hot methanol at 40-50° C., to which was added crushed ice, the mass stirred for 20 to 40 minutes, scratched and filtered. The solid was washed with water and dried to obtain the salt. XRD pattern showed amorphous form of the compound.

% water: 3-5% (different batches)

### EXAMPLE 3

### Preparation of Amorphous Form of Rimonabant Hydrochloride

1 g amorphous Rimonabant hydrochloride in partially hydrated form obtained as above was dried at 105-110° C. in a drier for about 4 to 5 hours to get the amorphous compound in anhydrous form.

### EXAMPLE 4

### Preparation of Form (II) of Rimonabant Hydrochloride

5 g of amorphous Rimonabant hydrochloride was stirred in diethyl ether at 25-30° C. for about 20 to 24 hours, filtered and washed with diethyl ether and subsequently removing the solvent from the filtered solid to get the solid compound. m.p. 237-244° C.

### EXAMPLE 5

### Preparation of Form (II) of Rimonabant Hydrochloride

5 g of amorphous Rimonabant hydrochloride was stirred in ethyl acetate at 25-30° C. for about 20 to 24 hours, filtered and washed with ethyl acetate and subsequently removing the solvent from the filtered solid to get the solid compound. m.p. 237-244° C.

### EXAMPLE 6

### Preparation of Form (III) of Rimonabant Hydrochloride

5 g of amorphous Rimonabant hydrochloride was stirred in isopropyl alcohol at 25-30° C. for about 20 to 24 hours, filtered and washed with isopropyl alcohol and subsequently removing the solvent from the filtered solid to get the solid compound. m.p. 244-245° C.

### EXAMPLE 7

### Preparation of Form (IV) of Rimonabant Hydrochloride

5 g of amorphous Rimonabant hydrochloride was stirred in 1-pentanol at 25-30° C. for about 20 to 24 hours, filtered and washed with 1-pentanol and subsequently removing the solvent from the filtered solid to get the solid compound. m.p. 242-244° C.

### EXAMPLE 8

### Preparation of Form (IV) of Rimonabant Hydrochloride

5 g of amorphous Rimonabant hydrochloride was stirred in acetone at 25-30° C. for 20 to 24 hours, filtered and washed with acetone and subsequently removing the solvent from the residue to get the solid compound. m.p. 243-247° C.

### EXAMPLE 9

### Preparation of Form (I) of Rimonabant Hydrochloride

1 g of amorphous Rimonabant hydrochloride was taken in ethyl acetate, heated on a water bath to 50-60° C. for 15-20 minutes and filtered. The solid was washed with anhydrous diethyl ether and dried to obtain 800 mg of the product, m.p. 218-220° C., % purity 99.72%.

Thus, the amorphous form of Rimonabant hydrochloride may be used to prepare the Form I of Rimonabant hydrochloride with very high purity.

### Stability of Different Crystalline Forms of Rimonabant Hydrochloride Prepared According to the Present Invention:

The crystalline forms were found to be stable in 5 month stability studies carried out at 25° C. 60% RH as well as 40° C., 75% RH. No polymorphic changes were observed. The amorphous forms were found to be stable at 3 months at room temperature.

### Solubility of Different Forms of Rimonabant Hydrochloride Prepared According to the Present Invention:

All the forms were soluble in alcohols such as methanol, ethanol etc. (at R.T.)
Form II is additionally partly soluble in acetone and acetonitrile (at R.T.)
Form III is additionally partly soluble in acetonitrile (at R.T.)
Form IV is additionally partly soluble in acetone and acetonitrile (at R.T.)

### Advantages of the Present Forms:

1. The amorphous forms of Rimonabant hydrochloride may be used to prepare the different crystalline forms of Rimonabant hydrochloride in pure forms.
2. The high melting nature of the crystalline forms from those reported earlier and their stability data indicates that the crystalline forms may have beneficial effects in formulation development.
3. The crystalline forms were easy to purify, stable and easily reproducible and easy to scale up at production level. Hence, these forms are of commercial significance.

1. Amorphous Rimonabant hydrochloride.

2. Amorphous Rimonabant hydrochloride as claimed in claim 1 which is further characterized by X-ray diffraction pattern substantially as depicted in FIG. 1.

3. A novel crystalline polymorph of Rimonabant hydrochloride characterized by X-ray diffraction patterns substantially as depicted in FIG. 2.

4. A novel crystalline polymorph of Rimonabant hydrochloride as claimed in claim 3, characterized by X-ray diffraction pattern with peaks at about 10.16, 11.54, 11.96, 13.49, 14.2, 14.51, 14.93, 15.4, 16.30, 16.53, 16.92, 17.50, 18.04, 18.29, 18.94, 19.69, 20.60, 21.12, 21.533, 21.97 22.33, 23.36, 23.86, 24.26, 24.85, 25.15, 25.57, 26.20, 26.66, 27.03, 27.43, 28.50, 29.20, 29.52, 30.36, 30.82, 31.40, 32.64, 34.88±0.2 degrees two-theta.

5. A novel crystalline polymorph of Rimonabant hydrochloride characterized by X-ray diffraction pattern substantially as depicted in FIG. 3.

6. A novel crystalline polymorph of Rimonabant hydrochloride as claimed in claim 5, characterized by X-ray diffraction pattern with peaks at about 12.85, 13.45, 14.40, 16.33, 17.59, 18.32, 18.92, 19.48, 19.87, 20.56, 20.94, 23.03, 23.88, 24.77, 25.31, 26.56, 27.42, 28.09, 28.59, 28.88, 29.76, 30.10, 30.36, 31.25, 32.52, 32.94, 33.95, 35.92, 37.09, 37.72±0.2 degrees two-theta.

7. A novel crystalline polymorph of Rimonabant hydrochloride characterized by X-ray diffraction pattern substantially as depicted in FIG. 4.

8. A novel crystalline polymorph of Rimonabant hydrochloride as claimed in claim 7, characterized by X-ray diffraction pattern with peaks at about 9.34, 10.15, 11.45, 13.48, 14.18, 14.51, 14.93, 15.40, 16.31, 16.52, 16.93, 17.50, 18.04, 18.30, 18.92, 19.67, 20.6, 21.14, 21.54, 21.96, 22.27, 23.34, 23.86, 24.22, 24.79, 25.10, 25.55, 26.23, 26.65, 27.01, 28.48, 29.19, 29.49, 30.34, 30.81, 31.38, 32.22, 32.63, 34.47, 34.85, 35.15, 38.94±0.2 degrees two-theta.

9. A process for the preparation of amorphous Rimonabant hydrochloride as claimed in claim 1 comprising:
a) dissolving/contacting Rimonabant base in suitable solvents selected from (C₁-C₆) alcohols, benzene, dichloromethane, dichloroethane, acetone, cyclohexane, dimethyl formamide, dimethyl acetamide, 1,4-dioxane, tetrahydrofuran or mixtures thereof;
b) treating with dilute HCl; and
c) stirring, removing the solvent, filtering and controlled drying the residue to obtain amorphous Rimonabant hydrochloride.

10. A process for the preparation of novel crystalline polymorph of Rimonabant hydrochloride as claimed in claim 3 comprising:
a) stirring of amorphous Rimonabant hydrochloride in ethyl acetate, suitable ethers selected from diethyl ether, methyl t-butyl ether, diisopropyl ether, or mixtures thereof; and
b) filtering and removing the solvent from the residue.

11. A process for the preparation of novel crystalline polymorph of Rimonabant hydrochloride as claimed in claim 5 comprising:
a) stirring of amorphous Rimonabant hydrochloride in isopropanol; and
b) filtering and removing the solvent from the residue.

12. A process for the preparation of novel crystalline polymorph of Rimonabant hydrochloride as claimed in claim 7 comprising:
a) stirring of amorphous Rimonabant hydrochloride in (C₅-C₁₂) alcohols, acetone or mixtures thereof; and
b) filtering and removing the solvent from the residue.

13. A process for preparing different crystalline forms of Rimonabant hydrochloride by dissolving the amorphous forms of Rimonabant hydrochloride of the present invention in appropriate solvents and subsequently separating the crystalline forms from the solution.

14. A pharmaceutical composition comprising the novel polymorphs of Rimonabant hydrochloride of the present invention as claimed in claim 1, comprising either single polymorph or their mixtures in combination with the pharmaceutically acceptable excipients.

15. A pharmaceutical dosage form comprising the pharmaceutical compositions containing novel polymorphs of Rimonabant hydrochloride of the present invention as claimed in claim 14.

16. Use of novel polymorphs of Rimonabant hydrochloride of the present invention or their pharmaceutically compositions as claimed in claim 1, for preparing medicaments suitable for the treatment of obesity, smoking cessation, overweight, Alzheimer's disease, Parkinson's disease and other related diseases in a mammal including human.

17. Method of treatment comprising administering to a person in need thereof, pharmaceutical compositions or pharmaceutically acceptable dosage forms containing the novel polymorphs of Rimonabant hydrochloride of the present invention, as claimed in claim 1 for the treatment of obesity, smoking cessation, overweight, Alzheimer's disease and Parkinson's disease.
